(19) United States
(12) Patent Application Publication
(10) Pub. No.: US 2017/0086906 A1
(43) Pub. Date: Mar. 30, 2017

(54) SURGICAL SYSTEM, MEDICAL DEVICE, AND CONTROL METHOD OF SURGICAL SYSTEM

(71) Applicant: OLYMPUS CORPORATION, Tokyo (JP)

(72) Inventor: Shoei TSURUTA, Tokyo (JP)

(73) Assignee: OLYMPUS CORPORATION, Tokyo (JP)

(21) Appl. No.: 15/378,455

(22) Filed: Dec. 14, 2016

Related U.S. Application Data
(63) Continuation of application No. PCT/JP2015/052994, filed on Feb. 3, 2015.

(30) Foreign Application Priority Data
Jun. 23, 2014 (JP) 2014-128530

(51) Int. Cl. A61B 18/10; A61B 1/05; A61B 90/98; A61B 90/00; A61B 1/018; A61B 90/96; A61B 17/34; A61B 18/08

(52) U.S. Cl. CPC A61B 18/10; A61B 17/3476; A61B 1/051; A61B 18/082; A61B 90/39; A61B 1/018; A61B 90/96; A61B 90/98; A61B 17/3498; A61B 2090/0807; A61B 2018/00708

(57) ABSTRACT

A surgical system includes: a treatment instrument including an insertion tube provided with a power receiver; a trocar including a power transmitter; and a power source device including a power source configured to output electric power to the trocar, wherein the treatment instrument includes a bar code that is an identifier located closer to a distal end of the insertion tube than the power receiver and indicating information of the treatment instrument, an information detector configured to acquire the information of the treatment instrument by detecting the bar code is located on the trocar, and when the inserted treatment instrument is a conforming treatment instrument, a control mode of the power source is shifted from a first standby mode that disables outputting drive power to a second standby mode that enables outputting the drive power.

### CROSS REFERENCE TO RELATED APPLICATION

This application is a continuation application of PCT/JP2015/052994 filed on Feb. 3, 2015 and claims benefit of Japanese Application No. 2014-128530 filed in Japan on Jun. 23, 2014, the entire contents of which are incorporated herein by this reference.

### BACKGROUND OF THE INVENTION

1. Field of the Invention

The present invention relates to a surgical system including a medical device configured to wirelessly receive electric power through an electromagnetic field, the medical device, and a control method of the surgical system.

2. Description of the Related Art

A trocar, which is an insertion assisting tool for inserting medical equipment such as a treatment instrument into a body of a subject, is retained in a body wall after insertion into an abdominal cavity, and the trocar is used as a guide tube of the treatment instrument configured to perform a treatment of biological tissue in the abdominal cavity.

In some cases, a cable is connected to the treatment instrument inserted into the body through an insertion hole of the trocar in order to supply electric power necessary for the treatment. It is preferable that no cable is used in order to improve operability in handling and the like when an operator operates the treatment instrument.

A surgical system configured to generate an alternating magnetic field from a power transmission coil of a trocar and wirelessly supply electric power to a power reception coil of a treatment instrument inserted into the trocar is disclosed in U.S. Pat. No. 6,371,967.

Here, unless there is special consideration and design in the insertion hole of the trocar, the treatment instrument that can be inserted is not limited to a treatment instrument with predetermined specifications including a power receiver configured to receive the electric power through the alternating magnetic field.

### SUMMARY OF THE INVENTION

An aspect of the present invention provides a surgical system including: a medical device including: an insertion tube provided with a power receiver configured to receive an electromagnetic field; and a treatment portion located on a distal end of the insertion tube and driven by electric power received by the power receiver; an insertion assisting tool provided with an insertion hole for inserting the insertion tube into a body of a subject and including a power transmitter configured to generate an electromagnetic field applied to the power receiver on an outer circumferential portion of the insertion hole; and a power source device including a power source configured to output electric power to the power transmitter, wherein the medical device includes an identifier located closer to a distal end of the insertion tube than the power receiver and indicating information of the medical device, an information detector configured to detect the identifier to detect insertion of the medical device into the insertion hole and acquire the information is located on the outer circumferential portion of the insertion hole of the insertion assisting tool, and when the medical device inserted into the insertion hole includes a power receiver receiving the electromagnetic field generated by the power transmitter based on a detection result of the information detector, a control mode of the power source is shifted from a first standby mode that disables outputting drive power for driving the treatment portion to the power transmitter to a second standby mode that enables outputting the drive power.

Another aspect provides a medical device of a surgical system including: the medical device including: an insertion tube provided with a power receiver configured to receive an electromagnetic field; and a treatment portion located on a distal end of the insertion tube and driven by electric power received by the power receiver; an insertion assisting tool provided with an insertion hole for inserting the insertion tube into a body of a subject and including a power transmitter configured to generate an electromagnetic field applied to the power receiver on an outer circumferential portion of the insertion hole; and a power source device including a power source configured to output electric power to the power transmitter, wherein an identifier located closer to a distal end of the insertion tube than the power receiver and indicating information of the medical device is included, an information detector configured to detect the identifier to detect insertion of the medical device into the insertion hole and acquire the information is located on the outer circumferential portion of the insertion hole of the insertion assisting tool, and when the medical device inserted into the insertion hole includes a power receiver receiving the electromagnetic field generated by the power transmitter based on a detection result of the information detector, a control mode of the power source is shifted from a first standby mode that disables outputting drive power for driving the treatment portion to the power transmitter to a second standby mode that enables outputting the drive power.

Another aspect provides a control method of the surgical system including: a medical device including: an insertion tube provided with a power receiver configured to receive an electromagnetic field; and a treatment portion located on a distal end of the insertion tube and driven by electric power received by the power receiver; an insertion assisting tool provided with an insertion hole for inserting the insertion tube into a body of a subject and including a power transmitter configured to generate an electromagnetic field applied to the power receiver on an outer circumferential portion of the insertion hole; and a power source device including a power source configured to output electric power to the power transmitter, wherein when the medical device inserted into the insertion hole includes a power receiver configured to receive the electromagnetic field generated by the power transmitter, a controller changes a control mode of the power source from a first standby mode at activation that disables outputting drive power for driving the treatment power to the power transmitter to a second standby mode that enables outputting the drive power.

### BRIEF DESCRIPTION OF THE DRAWINGS

FIG. 1 is a schematic diagram for describing a use state of a surgical system of a first embodiment;

FIG. 2 is a side view of a treatment instrument of the first embodiment;

FIG. 3 is a configuration diagram of the surgical system of the first embodiment;

FIG. 4 is a schematic diagram for describing sensing of an identifier of the surgical system of the first embodiment;

FIG. 5 is a flowchart for describing operation of the surgical system of the first embodiment;

FIG. 6A is a cross-sectional schematic diagram for describing operation of the surgical system of the first embodiment;

FIG. 6B is a cross-sectional schematic diagram for describing operation of the surgical system of the first embodiment;

FIG. 6C is a cross-sectional schematic diagram for describing operation of the surgical system of the first embodiment;

FIG. 7 is a cross-sectional schematic diagram for describing a surgical system of a first modification of the first embodiment;

FIG. 8 is a cross-sectional schematic diagram for describing a surgical system of a second modification of the first embodiment;

FIG. 9 is a cross-sectional schematic diagram for describing a surgical system of a third modification of the first embodiment;

FIG. 10A is a graph for describing position detection by the surgical system of the modifications of the first embodiment;

FIG. 10B is a graph for describing position detection by the surgical system of the modifications of the first embodiment; and

FIG. 11 is a cross-sectional schematic diagram for describing a configuration of a surgical system of a second embodiment.

### DETAILED DESCRIPTION OF THE PREFERRED EMBODIMENT(S)

### First Embodiment

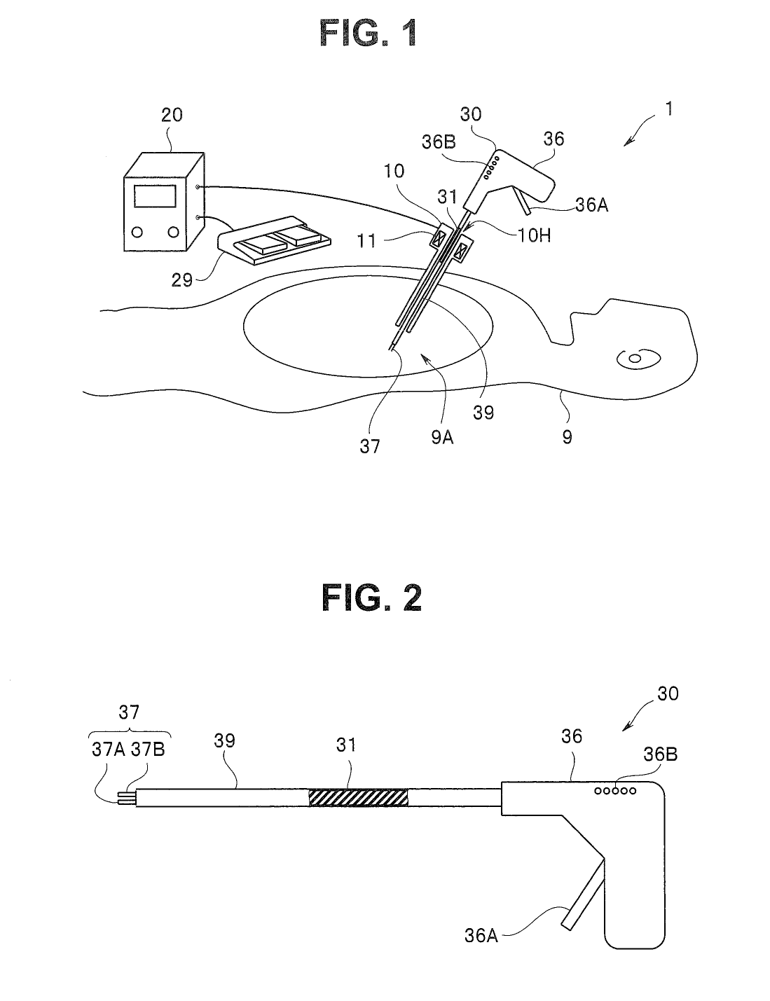

First, a surgical system 1 and a treatment instrument 30 that is medical equipment of a first embodiment will be described with reference to FIGS. 1 to 6C. Note that in the following description, drawings based on embodiments are schematic drawings, and a relationship (dimensional relationship) between thickness and width of constituent elements, a ratio of the thickness of respective parts, and the like are different from the reality. The dimensional relationship or the ratio between a plurality of drawings may also be different in some parts of the drawings.

The surgical system 1 includes a trocar 10 that is an insertion assisting tool, the treatment instrument 30, and a power source device 20. Note that although an endoscope or the like is also inserted into a body through another trocar in the surgical system 1, description and the like will be omitted.

As shown in FIG. 1, an insertion tube 39 of the treatment instrument 30 for surgery is inserted into a body 9A of a subject 9 through an insertion hole 10H of the trocar 10 punctured in a body wall of the subject 9.

As shown in FIG. 2, the treatment instrument 30 that is a high frequency treatment instrument includes: an operation portion 36; the elongated insertion tube 39 inserted into the body of the subject 9; and a treatment portion 37 located at a distal end of the insertion tube 39. An LED indicator 36B that is a notification section configured to notify a treatment state and the like to surgeon is located on the operation portion 36.

In the surgical system 1, electric power used for treatment is wirelessly supplied from the trocar 10 to the treatment portion 37 located on the distal end side of the insertion tube 39. Therefore, a cable for supplying electric power is not connected to the treatment instrument 30.

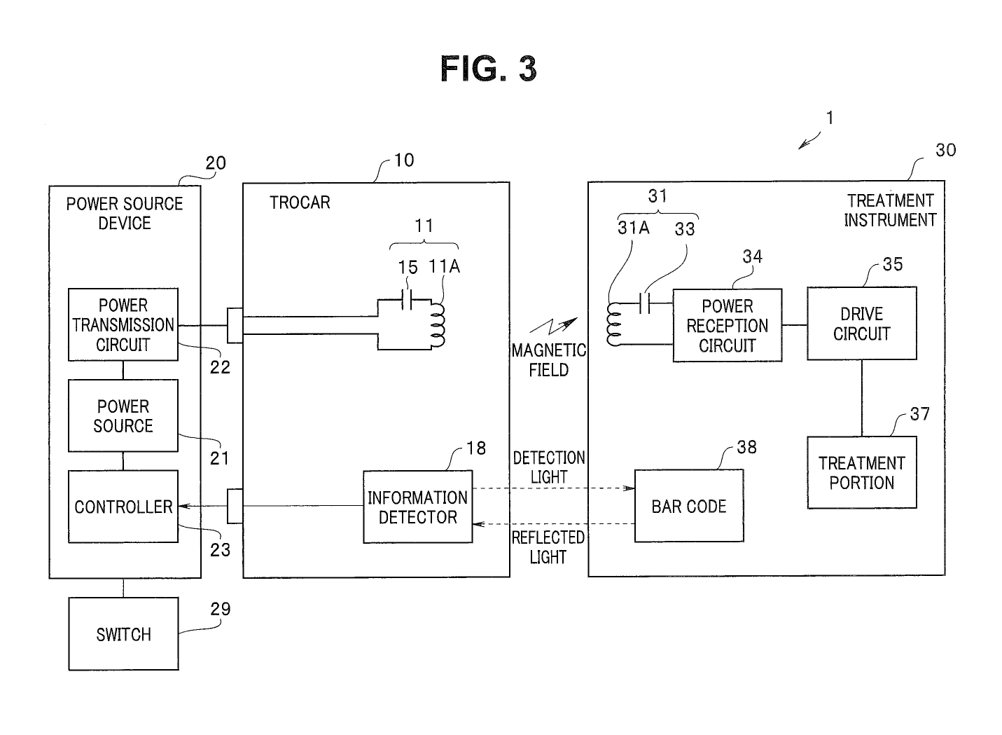

A power transmitter 11 is arranged on an outer circumferential portion of the insertion hole 10H of the trocar 10. The power transmitter 11 includes, for example, a solenoid power transmission coil 11A (see FIG. 3) and is wound around the outer circumferential portion of the insertion hole 10H. The power transmission coil 11A generates an alternating magnetic field when a drive signal is supplied from the power source device 20.

On the other hand, a power receiver 31 is located inside of the insertion tube 39 of the treatment instrument 30. The power receiver 31 is, for example, an elongated solenoid power reception coil 31A. When the insertion tube 39 is inserted into the insertion hole 10H of the trocar 10, the power reception coil 31A is electromagnetically coupled to the power transmission coil 11A and enters a state in which the magnetic field generated by the power transmission coil 11A can be received.

The treatment portion 37 configured to perform a treatment in the body 9A of the subject 9 includes a pair of jaws 37A and 37B that can be opened and closed. For example, an opened or closed state of the jaws 37A and 37B is linked with an opened or closed state of the handle 36A of the operation portion 36. The treatment portion 37 is not limited to a so-called treatment instrument. The treatment portion 37 can also be applied to various kinds of medical equipment including an electrical drive section driven by electric power, such as an image pickup device like a CCD and an illumination apparatus like an LED as the electrical drive section. A rigid endoscope and the like can also be preferably used as the medical equipment of the present invention.

When the operator turns on a switch 29 (trigger ON) in a state that a tissue to be treated is placed between the jaws 37A and 37B, the power transmission coil 11A generates an alternating magnetic field, and the power reception coil 31A receives power from the alternating magnetic field. Based on the received power, a high frequency current is applied to the jaws 37A and 37B, and a treatment, such as dissection, hemostasis, or the like is performed.

FIG. 3 shows a configuration of the surgical system 1. The power source device 20 outputs, for example, a high frequency power with a frequency equal to or higher than 100 kHz and equal to or lower than 100 MHz. It is preferable that the frequency of the high frequency power is selected from frequencies whose usage is admitted by laws or the like, so the frequency is, for example, 13.56 MHz. Although amplitude of the high frequency power is not particularly limited, it is preferable that a waveform is a sine wave because a general-purpose power source can be used.

As described previously, the power transmitter 11 of the trocar 10 generates an alternating magnetic field when alternating current power is supplied from the power source device 20 based on the operation of the switch 29 by the operator. The power transmitter 11 includes the solenoid power transmission coil 11A wound around the outer circumferential portion of the insertion hole 10H and a power transmission capacitor 15. A length of the power transmission coil 11A is, for example, from 10 mm to 50 mm.

The power transmission coil 11A and the power transmission capacitor 15 of the power transmitter 11 are connected in series and form a power transmission side LC series resonant circuit configured to generate an alternating magnetic field with a predetermined resonance frequency FR1. The power source device 20 outputs the alternating current power with the resonance frequency FR1. Note that a stray capacitance of the power transmission coil 11A may be utilized in place of the power transmission capacitor 15. A power transmission circuit 22 includes an impedance matching circuit (not shown) configured to perform impedance matching of a power source 21 and the resonant circuit.

Note that in the surgical system 1 shown in FIG. 3, the power transmission capacitor 15 is located on the trocar 10, and the power transmission circuit 22 is located on the power source device 20. However, the power transmission capacitor 15 and the power transmission circuit 22 may be located on the trocar 10 or may be located on the power source device 20.

The treatment instrument 30 includes the power receiver 31, a power reception circuit 34, a drive circuit 35, and the treatment portion 37. The power receiver 31 is electromagnetically coupled to the power transmission coil 11A of the power transmitter 11 of the trocar 10 and includes the power reception coil 31A configured to wirelessly receive electric power through the alternating magnetic field.

The power reception coil 31A is an elongated solenoid coil arranged in a major axis direction of the elongated cylindrical insertion tube 39, and a center axis of the power reception coil 31A substantially coincides with a center axis of the insertion tube. A length of the power reception coil 31A is, for example, equal to or greater than 100 mm and equal to or smaller than 200 mm so that part of the power reception coil 31A is inserted into the power transmission coil 11A during the treatment, and the power reception coil 31A may have a length such that the power reception coil 31A is located throughout an overall length of, for example, 300 mm of the insertion tube 39. That is, it is preferable that the length of the power reception coil 31A is longer than the length of the power transmission coil 11A. As a result, even when the treatment instrument 30 moves back and forth inside of the insertion hole 10H during the treatment, the power reception coil 31A can receive power.

A power reception capacitor 33 is connected in series to the power reception coil 31A to form a power reception side LC series resonant circuit configured to efficiently receive an alternating magnetic field with a predetermined resonance frequency FR2. The resonance frequency FR2 of the power reception side LC series resonant circuit is substantially the same as the resonance frequency FR1 of the power transmission side LC series resonant circuit, and electric power is efficiently and wirelessly transmitted and received in the surgical system 1 based on a phenomenon of magnetic resonance. Note that as described previously, the resonance frequencies FR1 and FR2 can be appropriately selected in the range of, for example, 100 kHz to 100 MHz.

Note that a stray capacitance of the power reception coil 31A may be utilized in place of the power reception capacitor 33. The power reception circuit 34, for example, rectifies an alternating current signal received by the power reception coil 31A to convert the alternating current signal to a direct current signal and smooths the signal. A DC/DC converter further adjusts the signal to a voltage to be supplied to the drive circuit 35. The power reception circuit 34 includes an impedance matching circuit (not shown) configured to perform impedance matching of the drive circuit 35 and the resonant circuit. The drive circuit 35 converts the electric power from the power reception circuit 34 to electric power suitable for driving the treatment portion 37 and outputs the electric power. For example, a drive signal with a frequency of 350 kHz and a voltage of 200 Vpp used for a treatment, such as dissection and coagulation, is supplied from the drive circuit 35 to the treatment portion 37 of the high frequency treatment instrument.

Note that although the power transmission circuit 22 and the power reception circuit 34 foul′ the resonant circuit in the description above, the resonance capacitor and the resonance operation are not essential configurations. The impedance matching circuit configured to perform impedance matching of the drive circuit 35 and the resonant circuit can also be installed behind the drive circuit 35 depending on the configuration.

In the surgical system 1 of the present embodiment, the treatment instrument 30 includes a bar code 38 which is a portion to be identified, that is, an identifier, located closer to the distal end of the insertion tube 39 than the power receiver 31. The bar code 38 is a mark including a plurality of striped patterns 38A to 38Z indicating information of the treatment instrument 30, such as a serial number and an amount of treatment power. The bar code 38 is formed by ring-shaped striped patterns circling around an outer circumferential portion of the insertion tube 39 so that the bar code 38 can be detected regardless of a rotation state of the insertion tube 39.

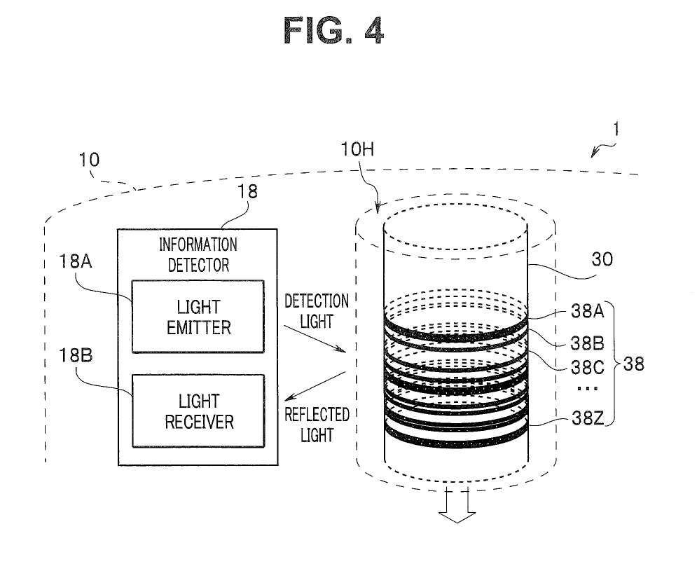

An information detector 18 configured to detect the bar code 38 to detect insertion of the treatment instrument 30 into the insertion hole 10H and acquire the information of the treatment instrument 30 is located on an outer circumferential portion of the insertion hole 10H of the trocar 10. As shown in FIG. 4, the information detector 18 includes: a light emitter 18A including an LED or the like configured to generate detection light for illuminating the bar code 38; and a light receiver 18B including a photodiode or the like configured to detect reflected light from the bar code 38.

Note that the identifier that is a portion to be identified is not limited to the bar code 38. The identifier may be a bar code formed by a striped pattern parallel to a longitudinal direction of the insertion tube 39 or may be a two-dimensional bar code. The identifier may magnetically record the information like an encoder or may electronically record the information like an RF-ID tag. A form of the information detector 18 is appropriately selected according to a form of the identifier.

A controller 23 of the power source device 20 includes a CPU or the like configured to sense insertion of the treatment instrument into the insertion hole 10H based on a detection result of the information detector 18. The controller 23 further senses whether the inserted treatment instrument is the treatment instrument 30 conforming to specifications of the trocar 10 including the power receiver 31 configured to receive an electromagnetic field generated by the power transmitter 11. When the treatment instrument conforms to the trocar 10, the controller 23 changes a control mode of the power source 21 from a first standby mode that disables outputting drive power for driving the treatment portion 37 to the power transmitter 11 to a second standby mode that enables outputting the drive power.

In the first standby mode, the drive power is not outputted from the power source 21 even if the operator turns on the switch 29.

When there is no partner of power receiver for appropriate electromagnetic coupling, the drive power is not outputted from the power source 21 in the surgical system 1 with the configuration, and the alternating magnetic field is not generated from the power transmission coil in a poor efficiency state. An excessive electric current does not flow in the power transmission coil. Therefore, the power transmitter does not generate heat, and electromagnetic field leakage does not occur. The treatment instrument 30 that is medical equipment of the surgical system 1 includes the bar code 38 indicating the information of the treatment instrument, and the alternating magnetic field is not generated from the power transmission coil 11A of the trocar 10 in a poor efficiency state. According to a control method of the surgical system 1, the alternating magnetic field is not generated from the power transmission coil in a poor efficiency state.

Note that as described later, when the power receiver 31 being at a position that allows receiving the electromagnetic field generated by the power transmitter 11 is not sensed in the surgical system 1, it is preferable that the controller 23 performs control to prevent outputting the drive power even if the power source 21 is in the second standby mode.

For example, the power source 21 in the second standby mode can output detection power smaller than the drive power to the power transmitter 11, and the controller 23 can sense that the power receiver 31 is at a position that allows receiving the electromagnetic field generated by the power transmitter 11 based on a change in electrical characteristics, such as an impedance of the power transmitter 11 and a phase between the current and the voltage.

In the surgical system 1, the controller 23 may further control an electric power value of the drive power outputted by the power source 21 based on the information acquired by the information detector 18.

When the controller 23 senses an abnormality during the treatment, the surgical system 1 may also change the control mode of the power source 21 to the first standby mode, that is, stop outputting the drive power.

### <Operation of Surgical System 1>

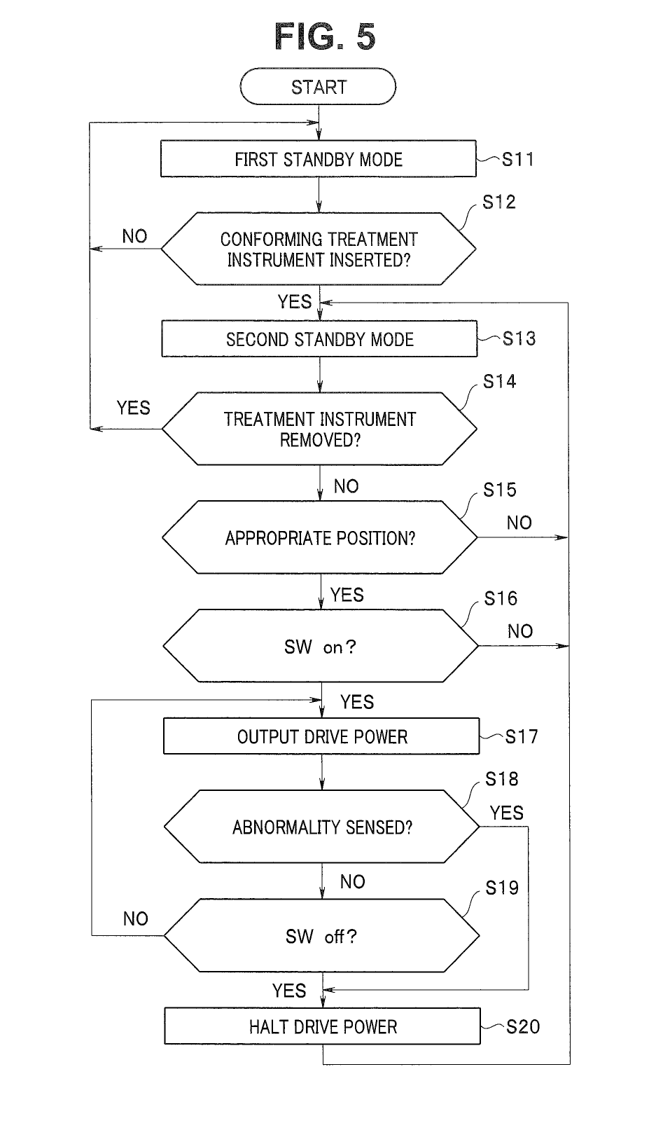

Next, operation of the surgical system 1 will be described along with a flowchart shown in FIG. 5.

### <Step S11> First Standby Mode (Activation Step)

The trocar 10 inserted into the subject 9 is connected to the power source device 20. Consequently, the electric power is supplied to the information detector 18 of the trocar 10. The control mode of the power source 21 at activation is the first standby mode that disables outputting the drive power. In the first standby mode, the drive power is not outputted from the power source 21 even if, for example, the operator accidentally turns on the switch 29.

Note that although the switch 29 is a foot switch separate from the power source device 20, the switch 29 operated by the operator may be located on the power source device 20, the trocar 10, or the treatment instrument 30.

### <Step S12> Identifier Detection Step

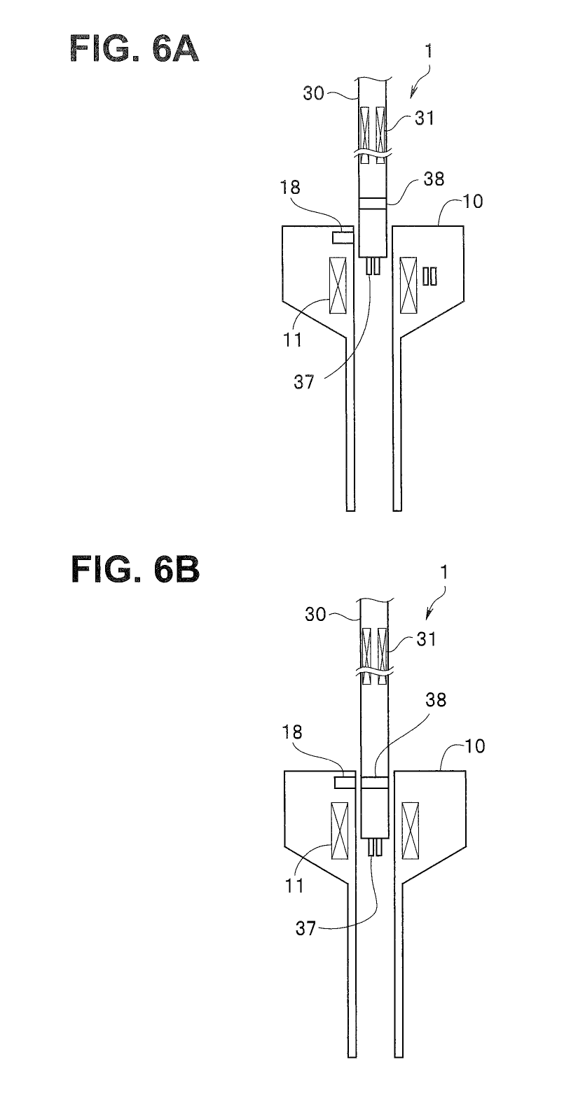

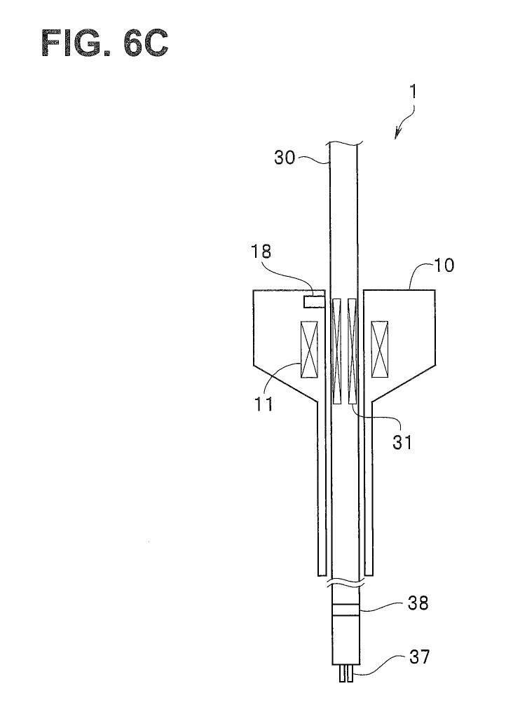

As shown in FIG. 6A, the insertion tube 39 of the treatment instrument 30 is inserted into the insertion hole 10H of the trocar 10. As shown in FIG. 6B, when the information detector 18 detects the bar code 38 located on the insertion tube 39, the detection result is transferred to the controller 23. For example, the information detector 18 just transfers a change in the electrical signal detected by the light receiver 18B to the controller 23, and the controller 23 including a CPU or the like analyzes and senses the information of the bar code 38.

### <Step S13> Second Standby Mode (Drive Power Output Enabling Step)

If the treatment instrument inserted into the insertion hole 10H conforms to the trocar 10 (S12: YES), the controller 23 changes the control mode of the power source 21 from the first standby mode that disables outputting the drive power for driving the treatment portion 37 to the power transmitter 11 to the second standby mode that enables outputting the drive power.

If the treatment instrument inserted into the insertion hole 10H does not conform to the trocar 10 (S12: NO), the controller 23 does not change the control mode of the power source 21 from the first standby mode.

Note that when the bar code 38 includes information of a treatment power value of the treatment instrument 30, it is preferable that the controller 23 controls the electric power outputted by the power source 21 to an electric power value according to the treatment power value of the treatment instrument 30.

It is also preferable that the controller 23 sets the output power of the power source 21 calculated from the treatment power value of the treatment instrument 30 as an upper limit output power value and controls the output power to a safe side to prevent operation of the power source 21 for outputting drive power equal to or greater than the upper limit output power value even if the operator accidentally makes an attempt to output the drive power equal to or greater than the upper limit output power value.

For example, when the treatment instrument 30 needs electric power of 50 W, power transmission and reception efficiency is taken into account to control the power source 21 to output electric power of 60 W, and the upper limit output power value is set to 70 W. Similarly, when the treatment instrument 30 needs electric power of 10 W, the upper limit output power value is set to 15 W, and the power source 21 outputs electric power of 12 W. When the treatment instrument 30 needs electric power of 1 W, the upper limit output power value is set to 1.5 W, and the power source 21 is controlled to output electric power of 1.2 W.

In the surgical system 1 in which the drive power is outputted according to the treatment power value of the treatment instrument 30, the operator does not have to operate the setting of the power source 21 according to the treatment instrument 30, and operability is excellent.

### <Step S14> Treatment Instrument Removal Detection Step

The information detector 18 can sense removal of the treatment instrument 30 from the insertion hole 10H by detecting again the bar code 38 detected once. For example, a mark indicating a direction is determined in advance in the bar code 38. The marks 38A and 38Z at both ends of the bar code 38 can be wider than the other marks, and the mark 38A can be a mark wider than the mark 38Z. In this way, the controller 23 can sense the removal based on the information detector 18 detecting the mark 38A after the mark 38Z.

If the removal of the treatment instrument 30 is sensed (S14: YES), the controller 23 returns the control mode of the power source 21 from the second standby mode to the first standby mode.

In the control method of the surgical system including step S14 (treatment instrument removal detection step), the drive power is not outputted even if the switch 29 is accidentally turned on although the treatment instrument is removed after completion of the treatment.

### <Step S15> Relative Position Detection Step

Although the power source 21 can output the drive power in the second standby mode, the power source 21 does not output the drive power unless at least the switch 29 is turned on. Furthermore, the power source 21 is controlled not to output the drive power even in the second standby mode in the surgical system 1 when the power receiver 31 being at a position that allows receiving the electric power through the alternating magnetic field generated by the power transmitter 11 is not sensed.

Note that although not shown in the flowchart, when the upper limit output power value is set, even if the power transmitter 11 and the power receiver 31 are not at most strongly coupled positions (even if the power receiver being at a position that allows receiving power at an optimal efficiency is not sensed), control may be performed to output the drive power by gradually changing the upper limit output power value according to the coupling state when the power transmitter 11 and the power receiver 31 being in a rather strongly coupled state is sensed (when the power receiver being at a position that allows receiving power at or more than a predetermined efficiency is sensed).

That is, as shown in FIG. 6C, simply put, the power receiver 31 of the treatment instrument 30 cannot be efficiently coupled to the alternating magnetic field generated by the power transmitter 11 before the power receiver 31 is inserted into the power transmitter 11.

When the mode becomes the second standby mode, the surgical system 1 enters a detection mode in which the power source 21 outputs the detection power to the power transmitter 11. The detection power may be sufficiently smaller than the drive power, such as about 10 mW.

In the detection mode, the controller 23 senses that the power receiver 31 is coupled to the alternating magnetic field generated by the power transmitter 11 and is at a position that allows receiving the electric power based on the change in the electrical characteristics of the power transmitter 11.

Note that the same detection mode as the mode for sensing the change in the electrical characteristics of the power transmitter 11 based on the detection power can also be used to locate the switch 29 operated by the operator on the treatment instrument 30. For example, the switch 29 is located on a path of the power reception circuit 34 of the treatment instrument 30, and the impedance on a load side as viewed from the power transmission circuit 22 provided with the detection signal significantly changes when an open state shifts to a conductive state. The controller 23 can wirelessly detect ON/OFF of the switch 29 located on the treatment instrument 30 based on the change in the electrical characteristics of the power transmission circuit 22.

As shown in FIG. 6C, when the power transmission coil 11A of the power transmitter 11 is electromagnetically coupled to the power reception coil 31A of the power receiver 31, the impedance on the load side detected from the side of the power transmission circuit 22 significantly decreases compared to when nothing is inserted. Based on the change in the electrical characteristics, the controller 23 can sense that the power receiver 31 is coupled to the alternating magnetic field generated by the power transmitter 11 and is at a position that allows receiving the electric power.

In the control method of the surgical system including step S15 (relative position detection step), maximum drive power is outputted only when the power transmitter 11 and the power receiver 31 are at relative positions that allow transmitting and receiving the electric power.

### <Step S16> SW ON (Treatment Step)

### <Step S17> Output Drive Power

In the state that the controller 23 senses that the power receiver 31 is at a position that allows appropriately coupling to the alternating magnetic field generated by the power transmitter 11 (S15: YES), the drive power is outputted from the power source 21 if the switch 29 is turned on (trigger ON) (S16: YES).

The treatment portion 37 performs the treatment based on the electric power of the power receiver 31 wirelessly received from the power transmitter 11.

### <Step S18> Abnormality Sensing Step

During the treatment, that is, when the switch 29 is turned on, the controller 23 can sense an abnormality of the treatment instrument 30 by detecting a change in the electrical characteristics, such as the impedance and the phase, on the load side detected from the side of the power transmission circuit 22. For example, when a disconnection occurs in the treatment portion 37, the impedance on the load side as viewed from the power transmission circuit 22 significantly increases.

When the controller 23 detects the abnormality, the controller 23 puts the power source 21 into the first standby mode and immediately halts outputting the drive power. It is preferable to further include a notification section configured to notify the operator of the abnormality. Examples of a notification method include causing the LED indicator 36B located on the operation portion 36 of the treatment instrument 30 to blink in red, displaying an abnormality message on a monitor displaying an endoscopic image not shown, and generating sound or light.

Note that in a surgical system including a plurality of treatment instruments and a plurality of trocars, notification sections can be provided on the treatment instruments or the trocars to immediately specify the treatment instrument in which an abnormality has occurred.

In the control method of the surgical system including step S16 (abnormality sensing step), the output of the drive power is automatically halted at the occurrence of the abnormality, or the output is significantly reduced. Therefore, a wrong treatment or the like caused by the abnormality is not performed.

### <Step S19> SW OFF (Treatment Ending Step)

### <Step S20> Halt Outputting Drive Power

When the switch 29 is turned off (trigger OFF), the power source 21 halts outputting the drive power. The power source 21 is controlled in the second standby mode of step S13 in preparation for a next treatment. Note that if the treatment instrument 30 is removed after the end of the treatment (S14: YES), the power source 21 is controlled in the first standby mode.

Note that it is only necessary that the surgical system 1 performs the operations of at least steps S11, S12, S13, S16, S17, S19, and S20. That is, the operations of steps S14, S15, and S18 are optional operations that are preferably performed in the surgical system 1.

### <Modifications>

Next, surgical systems 1A to 1C and treatment instruments 30B and 30C according to modifications of the first embodiment will be described. The surgical systems 1A to 1C and the treatment instruments 30B and 30C of the modifications are similar to the surgical system 1 and the treatment instrument 30. Therefore, the same reference signs are provided to the same components, and the description will not be repeated.

### <First Modification>

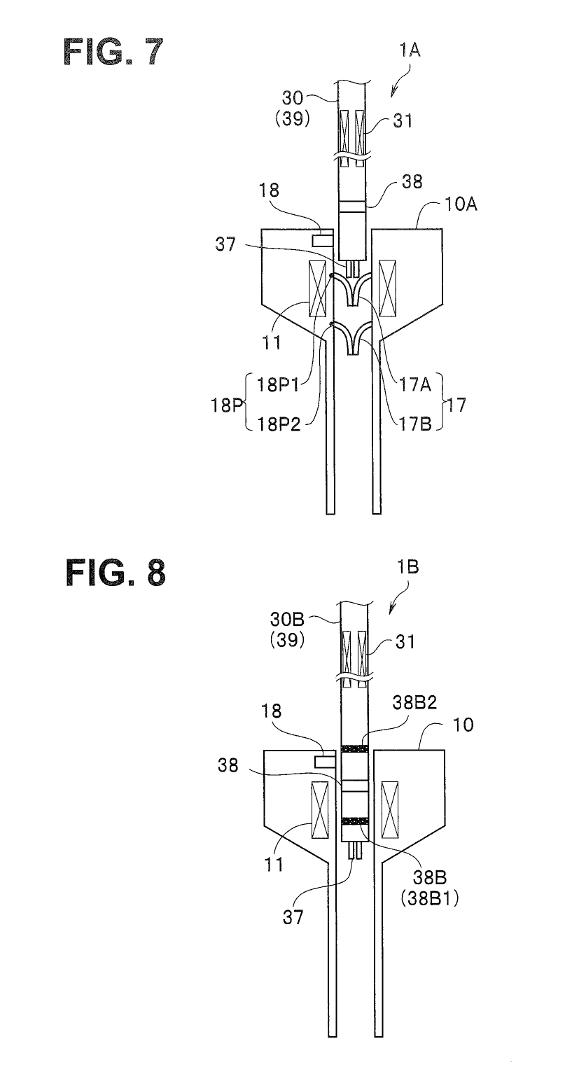

As shown in FIG. 7, sensors 18P (18P1, 18P2) located on check valves 17 (17A, 17B) of a trocar 10A detect insertion/removal of the treatment instrument 30 in the surgical system 1A of a first modification. The sensors 18P are, for example, pressure sensors configured to detect pressure applied to the check valves 17.

Note that although the description is omitted so far, the check valves 17 are also located on the trocar 10. To hold pressure in an abdominal cavity, the check valves 17 are airtight members made of silicone rubber or the like with a high elastic modulus located inside of the insertion hole 10H.

In the surgical system 1A, the insertion of the treatment instrument 30 is sensed when the sensor 18P1 and the sensor 18P2 sequentially detect application of the pressure. The removal of the treatment instrument 30 is sensed when the sensor 18P2 and the sensor 18P1 sequentially detect a decrease in the pressure.

In the surgical system 1A, the dedicated sensors 18P configured to detect the insertion/removal of the treatment instrument 30 are located on the trocar 10A. Therefore, the surgical system 1A can more surely detect the insertion/removal of the treatment instrument 30 compared to the surgical system 1.

### <Second Modification>

As shown in FIG. 8, the treatment instrument 30B of the surgical system 1B of a second modification detects the insertion/removal of the treatment instrument 30 based on designated marks 38B (38B1, 38B2) located on the insertion tube 39.

Although the marks 38B are ring-shaped black striped patterns circling around the outer circumferential portion of the insertion tube 39 just like the bar code 38, the marks 38B may be reflection members with a higher reflectance than the surroundings. The mark 38B1 may be a ring-shaped black striped pattern with a reflectance lower than the surroundings, and the mark 38B2 may be a ring-shaped reflection member.

In the surgical system 1B including the treatment instrument 30B, the designated marks 38B configured to detect the insertion/removal of the treatment instrument 30 are located on the treatment instrument 30. Therefore, the surgical system 1B can surely detect the insertion/removal of the treatment instrument 30.

### <Third Modification>

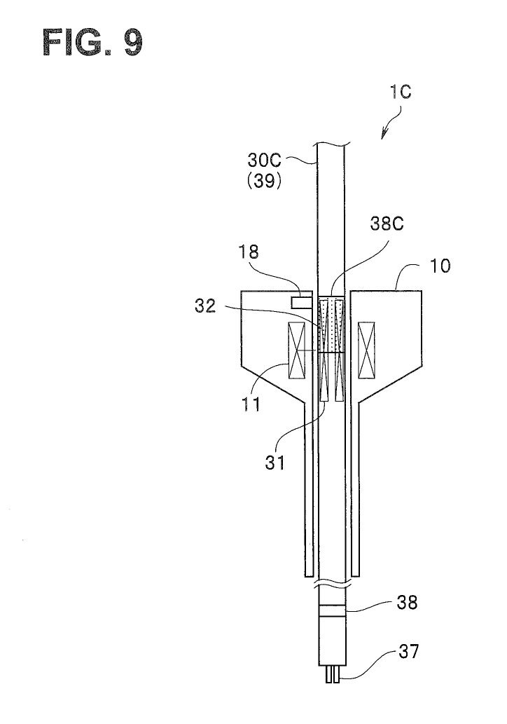

As shown in FIG. 9, in the treatment instrument 30C of the surgical system 1C of a third modification, the information detector 18 detects a reflection band 38C as a position mark indicating that the power receiver 31 is at a position that allows receiving the electric power through the alternating magnetic field generated by the power transmitter 11. Therefore, a ring-shaped reflection band 38C circling around the outer circumferential portion of the insertion tube 39 is located on the treatment instrument 30. A located position and a length of the reflection band 38C are set according to a positional relationship between the power transmitter 11 and the power receiver 31. For example, as shown in FIG. 9, the power transmitter 11 can be longer than the power receiver 31, and the electric power can be received even if the treatment instrument 30C is moved back and forth inside of the insertion hole 10H. The length of the reflection band 38C in this case is substantially equal to a length obtained by subtracting a length of the power transmitter 11 from a length of the power receiver 31. As for the positional relationship, the information detector 18 is positioned at a center portion of the reflection band 38C when the power transmitter 11 is at a center portion of the power receiver 31.

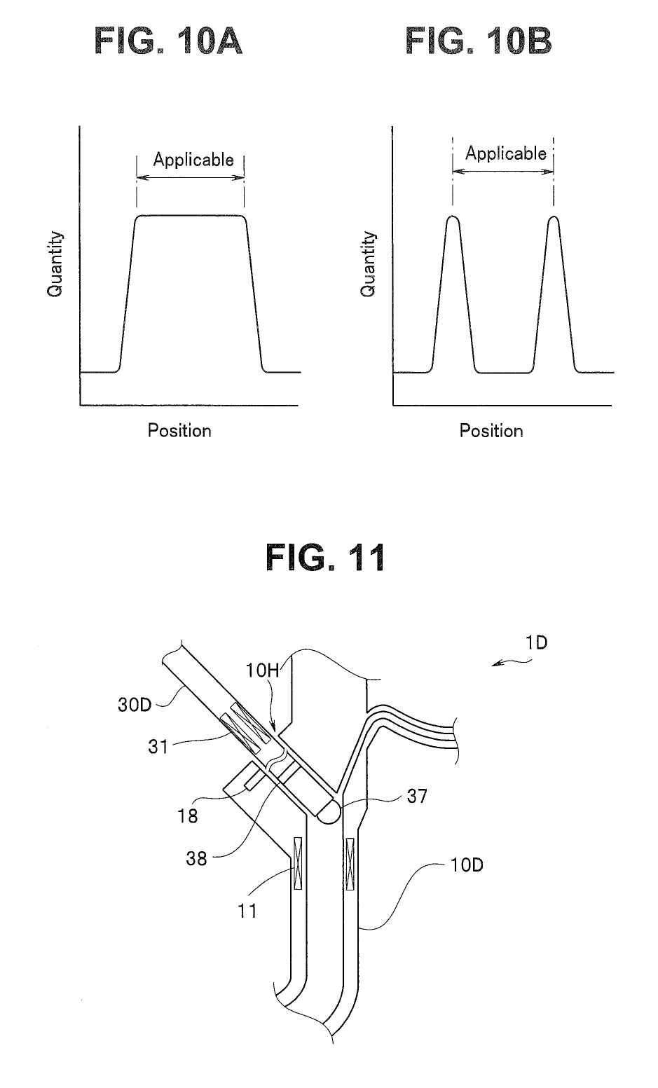

For example, as shown in FIG. 10A, a detection signal strength (Quantity) of the light receiver 18B of the information detector 18 is large when the power receiver 31 is at a position that allows receiving the electric power through the alternating magnetic field generated by the power transmitter 11. In FIGS. 10A and 10B, a horizontal axis indicates the position of the insertion tube 39 inside of the insertion hole 10H, that is, the relative position between the power transmitter 11 and the power receiver 31, and a vertical axis indicates the strength of the detection signal or the like. A range with a large detection signal strength (Quantity) is a range (Applicable) in which the power receiver 31 is at a position that allows receiving the electric power through the alternating magnetic field generated by the power transmitter 11. Note that the detection signal of the light receiver 18B is small when a ring-shaped black mark is included in place of the reflection band 38C.

Alternatively, two reflection bands 38C are located. As shown in FIG. 10B, the power receiver 31 is between positions where the detection signal of the light receiver 18B of the information detector 18 is strong, and the controller 23 senses that the power receiver 31 is at a position that allows receiving the electromagnetic field generated by the power transmitter 11.

In the surgical system 1C, the information detector 18 detects that the power receiver 31 is at a position that allows receiving the electric power through the alternating magnetic field generated by the power transmitter 11. Therefore, the configuration is simpler than the surgical system 1, and the control is easy.

Note that in the surgical systems 1B and 1C, the identifier is the bar code 38, a reflection portion, or the like, and the information detector 18 is an optical sensing portion in the cases described above. When the information detector 18 is a magnetic detection section or an electrical detection section, a dedicated identifier (portion to be identified) for detecting the insertion/removal of the treatment instrument 30 is used in order to generate the detection signal strength (Quantity) according to each detection mode.

### Second Embodiment

Next, a surgical system 1D and a treatment instrument 30D of a second embodiment as shown in FIG. 11 will be described. The operation and the like of the surgical system 1D and the treatment instrument 30D are similar to those of the surgical system 1 and the treatment instrument 30. Therefore, the same reference signs are provided to the components with the same functions, and the description will not be repeated.

In the surgical system 1D, the insertion assisting tool is a flexible endoscope 10D. In the flexible endoscope 10D, the channel (insertion tube) 10H configured to insert the treatment instrument 30D into the body of the subject can be assumed as an insertion hole.

The power transmitter 11 includes the power transmission coil 11A wound around the outer circumferential portion of the channel 10H of the flexible endoscope 10D.

As in the surgical systems 1, and 1A to 1C, the information detector 18 detects the bar code 38 or the like that is the identifier of the treatment instrument 30D to control the power source 21.

It is obvious that the surgical system 1D has the same effect as the surgical systems 1 and 1A to 1C.

Note that in the surgical system 1 and the like described above, the electric power is wirelessly supplied through the electromagnetic coupling based on the alternating magnetic field. On the other hand, it is obvious that a surgical system in which the electric power is wirelessly supplied through capacitive coupling based on the alternating magnetic field has the same effect as the surgical system 1 and the like.

The present invention is not limited to each of the embodiments and the like described above, and various changes, combinations, and applications can certainly be made without departing from the scope of the invention.

What is claimed is:

1. A surgical system comprising:
a medical device comprising:
an insertion tube provided with a power receiver configured to receive an electric power through an electromagnetic field;
a treatment portion located at a distal end of the insertion tube and driven by the electric power received by the power receiver; and
an identifier located closer to a distal end of the insertion tube than the power receiver and indicating information of the medical device;
an insertion assisting tool comprising:
an insertion hole for inserting the insertion tube into a body of a subject;
a power transmitter configured to generate an electromagnetic field applied to the power receiver on an outer circumferential portion of the insertion hole; and
an information detector configured to detect the identifier to detect insertion of the medical device into the insertion hole and detect the information is located on the outer circumferential portion of the insertion hole of the insertion assisting tool;
a power source device comprising a power source configured to output electric power to the power transmitter; and
a controller configured to change control mode of the power source between a first standby mode that disables outputting drive power for driving the treatment portion to the power transmitter and a second standby mode that enables outputting the drive power,
wherein the controller changes to the second standby mode from the first standby mode when the medical device inserted into the insertion hole comprises a power receiver receiving electric power through the electromagnetic field generated by the power transmitter as a detection result of the information detector.

2. The surgical system according to claim 1, wherein
when the power receiver being at a position that allows receiving the electric power through the electromagnetic field generated by the power transmitter is not sensed, the drive power is not outputted even if the control mode of the power source is the second standby mode.

3. The surgical system according to claim 1, wherein
when the power receiver being at a position that allows receiving the electric power at an optimal efficiency through the electromagnetic field generated by the power receiver is not sensed in the second standby mode, an upper limit output power value of the power source according to upper limit power set in advance in the medical equipment acquired as information of the medical device is changed.

4. The surgical system according to claim 1, wherein
when the information detector senses removal of the medical device from the insertion hole by detecting again the identifier detected once, the control mode of the power source returns from the second standby mode to the first standby mode.

5. The surgical system according to claim 1, wherein
the identifier is a mark formed by a ring-shaped striped pattern circling around the outer circumferential portion of the insertion tube, and
the information detector includes an optical detection section configured to detect the mark.

6. The surgical system according to claim 2, wherein
the power source in the second standby mode outputs detection power smaller than the drive power to the power transmitter, and the power receiver being at a position that allows receiving the electric power through the electromagnetic field generated by the power transmitter is sensed based on a change in electrical characteristics of the power transmitter.

7. The surgical system according to claim 2, wherein
a position mark for indicating an insertion position is located on an outer circumferential surface of the insertion tube of the medical device, and
the optical detection section detects the position mark to sense that the power receiver is at a position that allows receiving the electromagnetic field generated by the power transmitter.

8. The surgical system according to claim 1, wherein
an electric power value of the drive power is controlled based on the information acquired by the information detector.

9. The surgical system according to claim 1, wherein
the insertion assisting tool is a trocar, and
the medical device is a surgical treatment instrument.

10. The surgical system according to claim 1, wherein
the insertion assisting tool is an endoscope in which a channel including a hollow portion as the insertion hole is inserted into an insertion portion, and
the medical device is a treatment instrument inserted into the channel.

11. A medical device of the surgical system according to claim 1.

12. A control method of a surgical system comprising:
a medical device comprising:
an insertion tube provided with a power receiver configured to receive an electric power through an electromagnetic field;
a treatment portion located at a distal end of the insertion tube and driven by the electric power received by the power receiver; and
an identifier located closer to a distal end of the insertion tube than the power receiver and indicating information of the medical device;
an insertion assisting tool comprising:
an insertion hole for inserting the insertion tube into a body of a subject;
a power transmitter configured to generate an electromagnetic field applied to the power receiver on an outer circumferential portion of the insertion hole; and
an information detector configured to detect the identifier to detect insertion of the medical device into the insertion hole and detect the information is located on the outer circumferential portion of the insertion hole of the insertion assisting tool;
a power source device comprising a power source configured to output electric power to the power transmitter; and
a controller configured to change control mode of the power source between a first standby mode that disables outputting drive power for driving the treatment portion to the power transmitter and a second standby mode that enables outputting the drive power,
wherein the controller changes to the second standby mode from the first standby mode when the medical device inserted into the insertion hole comprises a power receiver receiving electric power through the electromagnetic field generated by the power transmitter as a detection result of the information detector.
